(19) United States
(12) Patent Application Publication
(10) Pub. No.: US 2015/0050377 A1
(43) Pub. Date: Feb. 19, 2015

(54) BIOMEDICAL 3D RAPID PROTOTYPING APPARATUS

(71) Applicant: MICROJET TECHNOLOGY CO., LTD, Hsinchu (TW)

(72) Inventors: KWO-YUAN SHI, HSINCHU (TW); KE-MING HUANG, Hsinchu (TW)

(73) Assignee: MICROJET TECHNOLOGY CO., LTD, Hsinchu (TW)

(21) Appl. No.: 13/969,150

(22) Filed: Aug. 16, 2013

(51) Int. Cl. B29C 67/00; A61L 9/20

(52) U.S. Cl. CPC B29C 67/0085; A61L 9/20; A61L 2430/00; B29K 2101/00
USPC 425/150

(57) ABSTRACT

A biomedical 3D rapid prototyping apparatus includes at least one bio-cartridge, a supply container, a construction platform, an identifying and monitoring device, a disinfection and temperature/humidity regulation device, a casing, and a negative pressure device. The bio-cartridge contains a bio-forming fluid, and has a printhead for ejecting the bio-forming fluid. The supply container stores a bio-building material. The bio-building material is spread in a construction chamber of the construction platform to form a construction layer, and the bio-cartridge is moved to the construction layer to print the bio-forming fluid on the construction layer, so that a cellular tissue is formed. The procedure of spreading the bio-building material and the procedure of printing the bio-forming fluid are repeatedly done, so that a transplantable living tissue or organ is quickly produced.

### FIELD OF THE INVENTION

The present invention relates to a biomedical 3D rapid prototyping apparatus, and more particularly to a biomedical 3D rapid prototyping apparatus for producing transplantable living tissues or organs.

### BACKGROUND OF THE INVENTION

As known, human organs may be suffered from damage because of accident or aging. When a patient's organ loses function or is overburdened, the patient may rely on organ transplantation to survive. However, in most situations, the patient may spend a lot of time in waiting for an appropriate donor organ. If no appropriate donor organ is found through the life, the illness condition cannot be improved and even the patient dies.

Conventionally, the organ transplantation technology may be divided into two major parts. A first major part of the organ transplantation technology is related to the transplantation of important human organs. However, the patients have to wait for the appropriate organs that are donated by donors with compatible physical characteristics. The waiting process is lengthy and usually torments the patients and their families. According to the current law system, the organs are only donated to the recipients on the waiting list. In addition, organ sales are illegal. Since the organs cannot be purchased, the preciousness is evident. Even if organs have been transplanted to the patients, the patients must periodically go back to hospital to be checked through their lives. In addition, the patients have to punctually take immunosuppressive drugs in order to prevent transplant rejection. That is, the follow-up treatment after the organ transplantation still torments the patients.

A second major part of the organ transplantation technology is related to the transplantation of traumas (e.g. skin transplantation). The donor skin is taken from a different site of the same patient or taken from other body. The donor skin taken from other body has a great tendency to cause rejection. In addition, the way of taking the donor skin from the same patient is imperfect because of “robbing Peter to pay Paul”.

From the above discussions, the low transplantation opportunity or the obvious transplant rejection problem makes the patients' wills become weaker and weaker. Particularly, if the immune systems of the patients attack the transplanted organ or tissue, the patients must behave with exceptional caution and on tenterhooks.

Therefore, it is an important issue to avoid the above drawbacks of the conventional organ transplantation technology.

### SUMMARY OF THE INVENTION

The present invention provides a biomedical 3D rapid prototyping apparatus. In the biomedical 3D rapid prototyping apparatus, a bio-forming fluid is printed on a human-compatible building material by a bio-cartridge. Consequently, a transplantable living tissue or organ is produced. In comparison with the conventional organ transplantation technology, the use of the biomedical 3D rapid prototyping apparatus of the present invention can increase the opportunity of implementing the organ transplantation or minimize the transplant rejection problem.

In accordance with an aspect of the present invention, there is provided a biomedical 3D rapid prototyping apparatus. The biomedical 3D rapid prototyping apparatus includes at least one bio-cartridge, a supply container, a construction platform, an identifying and monitoring device, a disinfection and temperature/humidity regulation device, a casing, and a negative pressure device. The bio-cartridge contains a bio-forming fluid, and has a printhead for ejecting the bio-forming fluid. The supply container stores a bio-building material. The construction platform includes a construction chamber, a chamber lift/lower mechanism, a movable feeding mechanism and a movable printing mechanism. The construction chamber is located over the chamber lift/lower mechanism and moved upwardly or downwardly to define a spreading space. The movable feeding mechanism is located over the construction chamber for supporting the bio-building material which is uniformly dropped down from the supply container and spreading the bio-building material in the spreading space to form a construction layer. The movable printing mechanism is disposed on the movable feeding mechanism for moving the bio-cartridge to perform a printing operation on the construction layer. The identifying and monitoring device is located over and beside the construction platform for identifying ejection points of the bio-forming fluid ejected from the printhead of the bio-cartridge, complementally ejecting the bio-forming fluid and monitoring an amount of the bio-forming fluid ejected from the printhead of the bio-cartridge. The disinfection and temperature/humidity regulation device is located over and beside the construction platform for sterilizing and disinfecting the construction platform, and providing a cell cultivation environment with appropriate temperature and humidity. The casing is used for covering the construction platform, so that the construction platform is in a sealed space. The negative pressure device is located under the construction platform for creating a negative pressure within the sealed space around the construction platform. The bio-building material is spread in the construction chamber to form the construction layer by the movable feeding mechanism, and the bio-cartridge is moved to the construction layer to print the bio-forming fluid on the construction layer, so that a cellular tissue is formed. The procedure of spreading the bio-building material and the procedure of printing the bio-forming fluid are repeatedly done, so that a transplantable living tissue or organ is quickly produced.

The above contents of the present invention will become more readily apparent to those ordinarily skilled in the art after reviewing the following detailed description and accompanying drawings, in which:

### BRIEF DESCRIPTION OF THE DRAWINGS

FIG. 1 is a schematic perspective view illustrating the outer appearance of a biomedical 3D rapid prototyping apparatus according to an embodiment of the present invention;

FIG. 2 is a schematic perspective view illustrating the inner structure of the biomedical 3D rapid prototyping apparatus of FIG. 1;

FIG. 3 is a schematic side view illustrating the inner structure of the biomedical 3D rapid prototyping apparatus of FIG. 1; and

FIG. 4 schematically illustrates a transplantable living tissue produced by the biomedical 3D rapid prototyping apparatus of FIG. 1.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

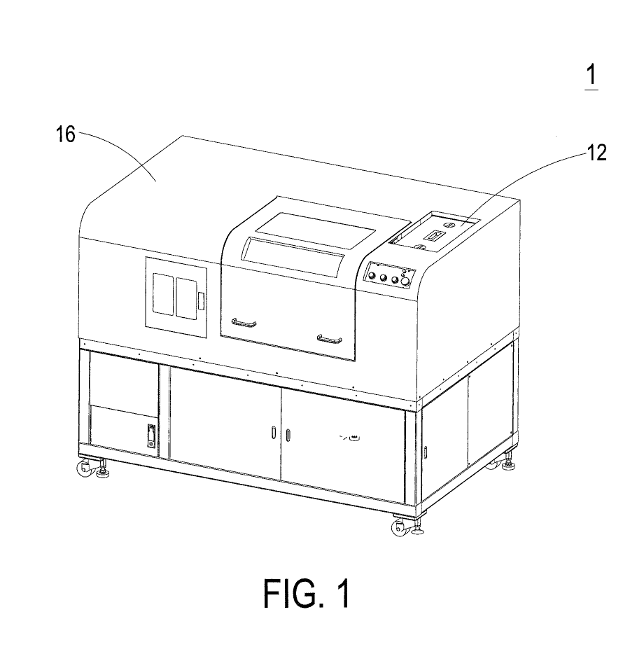

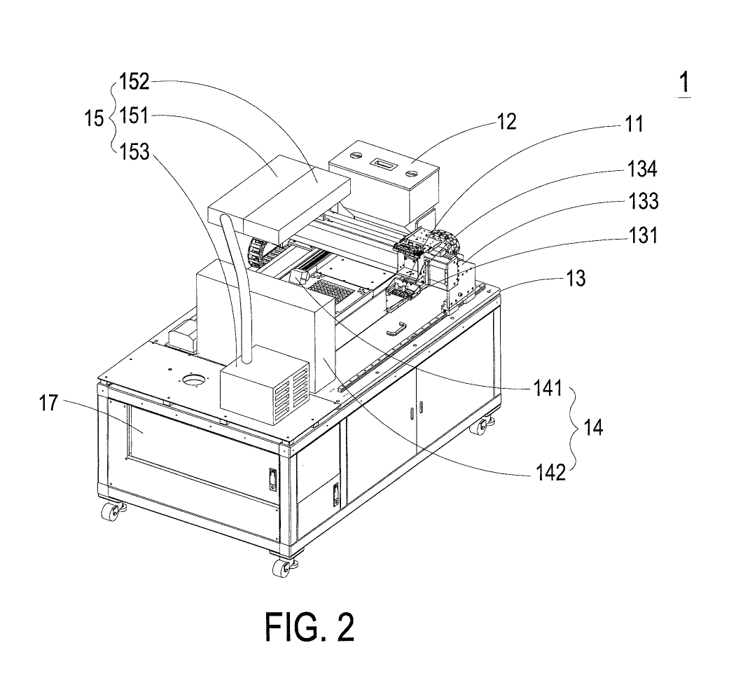

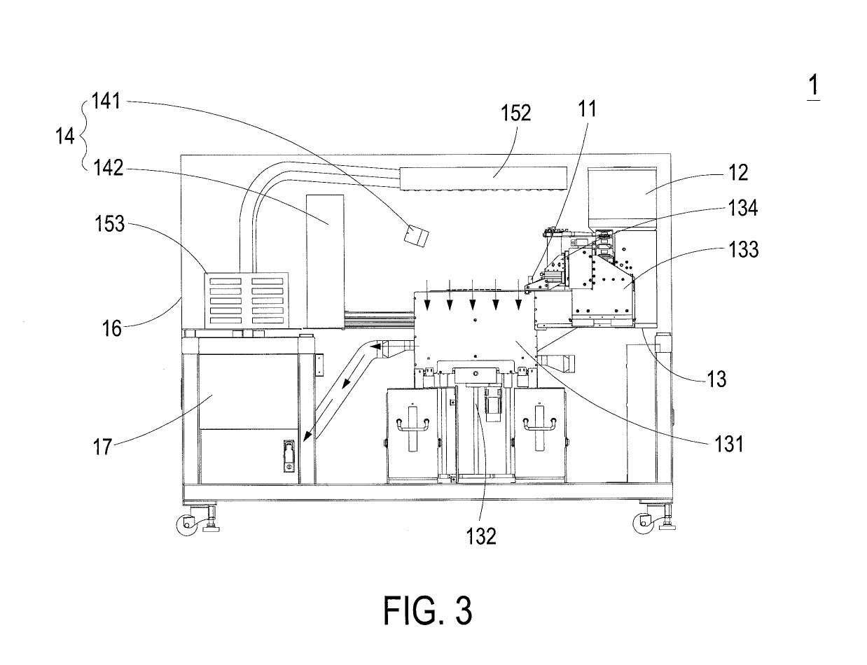

FIG. 1 is a schematic perspective view illustrating the outer appearance of a biomedical 3D rapid prototyping apparatus according to an embodiment of the present invention. FIG. 2 is a schematic perspective view illustrating the inner structure of the biomedical 3D rapid prototyping apparatus of FIG. 1. FIG. 3 is a schematic side view illustrating the inner structure of the biomedical 3D rapid prototyping apparatus of FIG. 1. As shown in FIGS. 1, 2 and 3, the biomedical 3D rapid prototyping apparatus 1 comprises at least one bio-cartridge 11, a supply container 12, a construction platform 13, an identifying and monitoring device 14, a disinfection and temperature/humidity regulation device 15, a casing 16, and a negative pressure device 17.

The bio-cartridge 11 contains a bio-forming fluid. The bio-forming fluid is obtained from mass cultivation of autologous cells (e.g. healthy stem cells) or composed of tissue cells of autologous organs or biological substrates of connective tissues. After the bio-forming fluid is introduced into the bio-cartridge 11, the bio-cartridge 11 is packaged in a non-toxic and sterile environment. Moreover, the bio-cartridge 11 has a printhead (not shown) for ejecting the bio-forming fluid.

The supply container 12 is used for storing a bio-building material. An example of the bio-building material includes but is not limited to an organ base material, a tissue base material, a connective tissue powder of a solid powdery collagen protein, a high-strength polymer, a metal ceramic powder or any other human-compatible building material. That is, the bio-building material is compatible with the bio-forming fluid, but it is not limited thereto.

The construction platform 13 comprises a construction chamber 131, a chamber lift/lower mechanism 132, a movable feeding mechanism 133, and a movable printing mechanism 134. The construction chamber 131 is located at a middle region of the construction platform 13, and located over the chamber lift/lower mechanism 132. Moreover, the construction chamber 131 is movable upwardly or downwardly to define a spreading space. The movable feeding mechanism 133 is disposed above the construction chamber 131 and movable along a first direction. Moreover, the movable feeding mechanism 133 can support the bio-building material that is uniformly dropped down from the supply container 12. When the movable feeding mechanism 133 is moved to the position over the construction chamber 131, the bio-building material is spread in the spreading space. Consequently, a construction layer to be printed is formed. The movable printing mechanism 134 is disposed on the movable feeding mechanism 133, and movable along a second direction, wherein the second direction is perpendicular to the first direction. The bio-cartridge 11 is supported on the movable printing mechanism 134. Consequently, the bio-cartridge 11 can be moved along the second direction to perform a printing operation on the construction layer.

The identifying and monitoring device 14 is located over and beside the construction platform 13. The identifying and monitoring device 14 comprises at least one identifying unit 141 and at least one monitoring unit 142. The identifying unit 141 is located over the construction platform 13 for identifying whether the bio-forming fluid is ejected from the printhead (not shown) of the bio-cartridge 11 and whether the ejection points are at the predetermined positions, thereby controlling the bio-cartridge 11 to perform an operation of complementally ejecting the bio-forming fluid. The monitoring unit 142 is located beside the construction platform 13 for monitoring whether the amount of the bio-forming fluid ejected from the printhead of the bio-cartridge 11 complies with the requirements.

The disinfection and temperature/humidity regulation device 15 is located over and beside the construction platform 13. The disinfection and temperature/humidity regulation device 15 comprises at least one anaerobic gas sterilization unit 151, at least one UV disinfection unit 152, and at least one temperature/humidity regulation unit 153. The anaerobic gas sterilization unit 151 and the UV disinfection unit 152 are located beside each other. In addition, the anaerobic gas sterilization unit 151 and the UV disinfection unit 152 are located over the construction platform 13 for sterilizing and disinfecting the construction platform 13. The temperature/humidity regulation unit 153 is located beside the construction platform 13 for providing a cell cultivation environment with appropriate temperature and humidity.

The casing 16 is used for covering a part of the construction platform 13 in order to cover and protect the bio-cartridge 11, the supply container 12, the construction platform 13, the identifying and monitoring device 14 and the disinfection and temperature/humidity regulation device 15. Consequently, the construction platform 13 is in a sealed space. Under this circumstance, a clean printing environment is provided to prevent pollution of foreign matters.

The negative pressure device 17 is located under the construction platform 13. During operation of the negative pressure device 17, the air within the region between the casing 16 and the construction platform 13 is exhausted to the surroundings. Consequently, the sealed space around the construction platform 13 has a weak negative pressure. In FIG. 3, the airflow path during operation of the negative pressure device 17 is indicated by arrows.

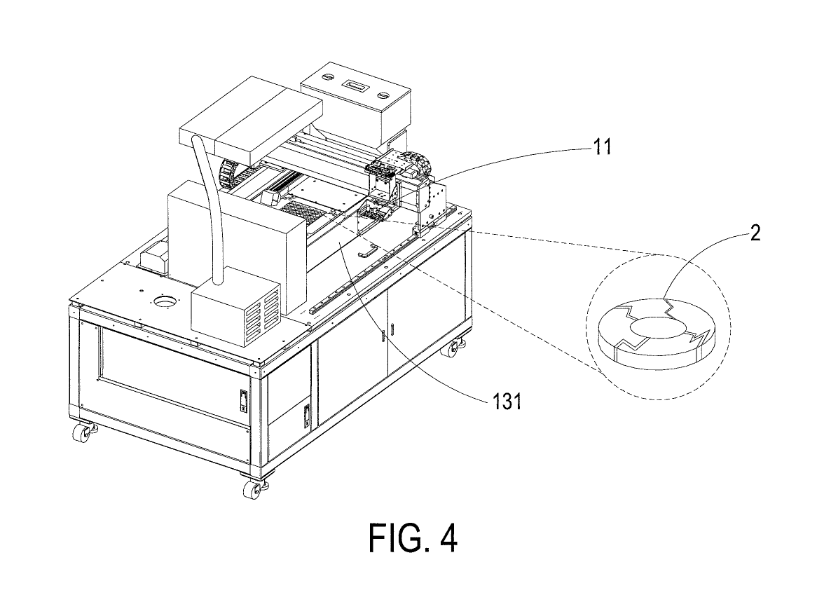

From the above discussions, biological tissues or organs in the biomedical field can be constructed by the biomedical 3D rapid prototyping apparatus 1. A process of constructing a tissue or organ by the biomedical 3D rapid prototyping apparatus will be illustrated in more details as follows. The bio-cartridge 11 is supported on the movable printing mechanism 134 and contains a bio-forming fluid. The movable feeding mechanism 133 is used to support the bio-building material that is uniformly dropped down from the supply container 12. The construction layer is spread in the construction chamber 131. The movable printing mechanism 134 is used to move the bio-cartridge 11 to the predetermined positions. The bio-forming fluid is ejected from the printhead of the bio-cartridge 11 so as to be printed on the construction layer. Consequently, the bio-forming fluid is formed on the construction layer. A resulting cellular tissue 2 is shown in FIG. 4. Next, the construction chamber 131 is lowered by the chamber lift/lower mechanism 132 so as to be moved for a certain displacement. The movable feeding mechanism 133 is moved to the position over the construction chamber 131 again, and the bio-building material is spread in the spreading space to form a second construction layer. Then, the bio-cartridge 11 is moved to the predetermined positions by the movable printing mechanism 134. The bio-forming fluid is ejected from the printhead of the bio-cartridge 11 so as to be printed on the second construction layer. Consequently, the bio-forming fluid is formed on the second construction layer, and an additional cellular tissue 2 is produced. The above procedures are repeatedly done to spread the bio-building material and print the bio-forming fluid in order to produce the cellular tissue 2. In such way, the desired living tissue or organ can be printed in a ply-by-ply stacking manner. The living tissue or organ produced by the biomedical 3D rapid prototyping apparatus can minimize the transplant rejection problem.

From the above descriptions, the present invention provides a biomedical 3D rapid prototyping apparatus. In the biomedical 3D rapid prototyping apparatus, a bio-forming fluid is printed on a human-compatible building material by the bio-cartridge. Consequently, a transplantable living tissue or organ is produced. In comparison with the conventional organ transplantation technology, the use of the biomedical 3D rapid prototyping apparatus of the present invention can increase the opportunity of implementing the organ transplantation or minimize the transplant rejection problem. Moreover, the identifying and monitoring device is used for identifying whether the ejection points of the bio-forming fluid ejected from the printhead of the bio-cartridge are at the predetermined positions, controlling the bio-cartridge to complementally eject the bio-forming fluid and monitoring whether the amount of the bio-forming fluid ejected from the printhead of the bio-cartridge complies with the requirements. Consequently, a cellular tissue can be produced. Moreover, the disinfection and temperature/humidity regulation device is used for sterilizing and disinfecting the construction platform, and providing a cell cultivation environment with appropriate temperature and humidity. Moreover, the negative pressure device is used for creating a negative pressure within the sealed space around the construction platform. Consequently, an appropriate cell cultivation environment with no cross-contamination is created. In other words, the biomedical 3D rapid prototyping apparatus of the present invention can be operated in a clean environment to print out a transplantable living tissue or organ.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A biomedical 3D rapid prototyping apparatus, comprising:
at least one bio-cartridge containing a bio-forming fluid, and having a printhead for ejecting the bio-forming fluid;
a supply container for storing a bio-building material;
a construction platform comprising a construction chamber, a chamber lift/lower mechanism, a movable feeding mechanism and a movable printing mechanism, wherein the construction chamber is located over the chamber lift/lower mechanism and moved upwardly or downwardly to define a spreading space, wherein the movable feeding mechanism is located over the construction chamber for supporting the bio-building material which is uniformly dropped down from the supply container and spreading the bio-building material in the spreading space to form a construction layer, wherein the movable printing mechanism is disposed on the movable feeding mechanism for moving the bio-cartridge to perform a printing operation on the construction layer;
an identifying and monitoring device located over and beside the construction platform for identifying ejection points of the bio-forming fluid ejected from the printhead of the bio-cartridge, complementally ejecting the bio-forming fluid and monitoring an amount of the bio-forming fluid ejected from the printhead of the bio-cartridge;
a disinfection and temperature/humidity regulation device located over and beside the construction platform for sterilizing and disinfecting the construction platform, and providing a cell cultivation environment with appropriate temperature and humidity;
a casing for covering the construction platform, so that the construction platform is in a sealed space;
a negative pressure device located under the construction platform for creating a negative pressure within the sealed space around the construction platform,
wherein the bio-building material is spread in the construction chamber to form the construction layer by the movable feeding mechanism, and the bio-cartridge is moved to the construction layer to print the bio-forming fluid on the construction layer, so that a cellular tissue is formed, wherein the procedure of spreading the bio-building material and the procedure of printing the bio-forming fluid are repeatedly done, so that a transplantable living tissue or organ is quickly produced.

2. The biomedical 3D rapid prototyping apparatus according to claim 1, wherein the bio-forming fluid is obtained from mass cultivation of autologous cells or composed of tissue cells of autologous organs or biological substrates of connective tissues.

3. The biomedical 3D rapid prototyping apparatus according to claim 1, wherein the bio-building material is an organ base material, a tissue base material, a connective tissue powder of a solid powdery collagen protein, a high-strength polymer, a metal ceramic powder or a human-compatible building material.

4. The biomedical 3D rapid prototyping apparatus according to claim 1, wherein the identifying and monitoring device comprises at least one identifying unit, wherein the identifying unit is located over the construction platform for identifying whether the bio-forming fluid is ejected from the printhead of the bio-cartridge and whether the ejection points are at the predetermined positions, thereby controlling the bio-cartridge to complementally eject the bio-forming fluid.

5. The biomedical 3D rapid prototyping apparatus according to claim 1, wherein the identifying and monitoring device comprises at least one monitoring unit, wherein the monitoring unit is located beside the construction platform for monitoring whether the amount of the bio-forming fluid ejected from the printhead of the bio-cartridge complies with requirements.

6. The biomedical 3D rapid prototyping apparatus according to claim 1, wherein the disinfection and temperature/humidity regulation device comprises at least one anaerobic gas sterilization unit, at least one UV disinfection unit and at least one temperature/humidity regulation unit, wherein the anaerobic gas sterilization unit and the UV disinfection unit are configured for sterilizing and disinfecting the construction platform, and the temperature/humidity regulation unit is configured for providing a cell cultivation environment with appropriate temperature and humidity.
